(19) United States
(12) Patent Application Publication
(10) Pub. No.: US 2011/0020840 A1
(43) Pub. Date: Jan. 27, 2011

(54) METHOD AND KITS FOR DETECTING ANTIBODIES AGAINST THERAPEUTIC ANTIBODIES

(76) Inventors: Gerrit Jan Wolbink, Amsterdam (NL); Steven Olivier Stapel, Amsterdam (NL)

Correspondence Address: HOFFMANN & BARON, LLP, 6900 JERICHO TURNPIKE, SYOSSET, NY, 11791, US

(21) Appl. No.: 12/812,690

(22) PCT Filed: Jan. 15, 2008
(86) PCT No.: PCT/NL08/50028

§ 371 Date: Oct. 6, 2010

(51) Int. Cl. G01N 33/53; G01N 33/543; G01N 33/544; G01N 33/553; G01N 33/545

(52) U.S. Cl. 435/7.9; 436/518; 436/529; 436/526; 436/528; 436/531

(57) ABSTRACT

The invention relates to the field of immunology and immunological diagnostics. More specifically, it relates to detection of the formation of antibodies in a subject that is treated with a therapeutic antibody. Provided is A diagnostic method for determining the presence of IgG antibodies against a therapeutic antibody in a subject, comprising the steps of: a) providing a solid carrier capable of binding the constant region of IgG antibodies; b) isolating a sample from a subject to be tested for the presence of IgG antibodies against a therapeutic antibody; c) incubating said carrier with said sample under conditions suitable for immobilizing IgG antibodies on said solid carrier; d) incubating said immobilized antibodies with an antigenic fragment of said therapeutic antibody under conditions that allow for complex formation between at least part of said immobilized antibodies and said antigenic fragment, said fragment lacking a constant region and being conjugated to a detectable label; and wherein said incubation is performed in the presence of an unlabeled antigenic fragment of a non-therapeutic antibody lacking a constant region. Also provided are kits for use in such a method.

The invention relates to the field of immunology and immunological diagnostics. More specifically, it relates to detection of the formation of antibodies in a subject that is treated with a therapeutic antibody.

There is a large and increasing number of therapeutic antibodies approved for clinical use and many more are undergoing preclinical studies and clinical trials in humans. Most of them are monoclonal antibodies (mAb), chimeric or ‘humanized’ antibodies, or fragments thereof. There are at least about twenty different therapeutic antibodies on the market and more than 150 are currently in clinical trials (1, 2). Indications for these therapeutics are varied and include, e.g., organ transplantation (OKT3®, Orthoclone®, Simulect®, Zenapax®), oncology (Rituxan®, Panorex®, Herceptin®, Mylotarg®, Campath®, Zevalin®, Bexxar®, Erbitux®, Avastin®, HuMax-CD4®), infectious disease (Synagis®), inflammation and autoimmune disease (Remicade®, Humira®, Amevive®, Enbrel®), multiple sclerosis (Tysabri®) and allergic asthma (Xolair®)). The therapeutic activity of such drugs may be mediated via different mechanisms of action, for example, by inhibiting signaling events in target cells, by direct induction of apoptosis, as well as by indirect immunologic mechanisms.

Clinical trials in rheumatoid arthritis (RA) have demonstrated that antibodies directed against the cytokine tumor necrosis factor α (TNF-α) (adalimumab [Humira®], infliximab [Remicade®]) are highly beneficial for most patients who are refractory to classic treatment with disease-modifying anti-rheumatic drugs, methotrexate or steroid therapy. These anti-inflammatory effects of infliximab have led to their use in other inflammatory diseases such as Crohn's disease and ankylosing spondylitis (AS), with a similar efficacy to that in RA.

However, a treatment with therapeutic antibodies (TA) can result in the formation of antibodies (ATA's) against the therapeutic antibodies administered. This may lead to unwanted side effects and loss of efficacy of the treatment. For example, in case of infliximab the presence of these antibodies has been associated with infusion reactions in 7-19% of patients, and they may also shorten the duration of the effect of infliximab when this is given repeatedly. Baert et al. (3) investigated the relation between antibodies to infliximab and post-infusion infliximab concentrations, the clinical effect of infliximab, and infusion-related side effects in patients with Crohn's disease. The present inventors reported that nearly half of the RA patients treated with infliximab developed anti-infliximab antibodies within the first year of treatment, and that the development of anti-infliximab antibodies was associated with a reduced response to treatment (4).

Antigenicity of therapeutic antibodies is observed for various types of therapeutic antibodies, and was found not to be dependent on the extent of humanization. It can be observed for murine IgG1 and IgG2a antibodies, as well as for chimeric IgG1 antibodies and humanized IgG1 antibodies. The incidence rate of autoantibodies to therapeutic antibodies differs significantly between individual therapeuticals, and ranges from about 80% for the murine IgG1 antibody OKT3® and 10-57% for the chimeric IgG1 antibody Remicade® to less than 2% for chimeric IgG1 antibody Simulect® or Campath®, a humanized IgG1 antibody (5).

Virtually all therapeutic proteins, including therapeutic antibodies, elicit some level of antibody response, which, in some cases, can lead to potentially serious side effects and loss of therapy responsiveness. The latter can be, at least partially, overcome by adjusting the dose. For example, it was found that in some patients with anti-infliximab antibodies showing inadequate response to treatment, continuation of the treatment with higher dosages of infliximab resulted in decreased levels of antibodies against the therapeutic antibody (4)

Thus, one of the major concerns in the area of therapeutic antibodies, despite of their wide usage, is potential development of anti-therapeutic antibodies which in return may interfere with therapy efficacy, as judged mainly by observing the relapse of signs and symptoms of disease and necessitate dose-increase or ending of the treatment.

The potential immunogenicity of therapeutic antibodies is a concern for clinicians and requires appropriate detection and quantitation of antibody development against therapeutic antibodies during treatment with established therapeutics and during clinical trials.

Current methods to monitor the presence of anti-therapeutic antibodies (ATA) in a patient receiving therapeutic antibody treatment typically involve enzyme-linked immunosorbent assay (ELISA)-based methodologies (Centocor standard operating procedure). However, a major disadvantage of these ELISA's may be their relatively high background signal, since tests for antibodies against antibodies are, especially in this format, prone to non-specific binding. Moreover, capturing of ATA's using immobilized TA may cause unwanted binding, via an Fc-Fc interaction, of non-ATA's in a patient's serum to TA. In particular, IgG4 antibodies and “rheuma factors” (IgM, IgG, IgA), give rise to high background values. This problem is not encountered in an ELISA format wherein only the F(ab′)2 fragment of a therapeutic monoclonal antibody is used to capture ATA's. However, a drawback of this of assay resides in the fact that any serum, be it of a healthy control or a patient receiving TA treatment, may contain IgG antibodies that bind to F(ab′)2, irrespective of the nature of the immobilized F(ab′)2, thereby also causing a high background signal.

In view of the above, it is a goal of the present invention to provide an improved diagnostic test that allows for reliable and sensitive detection of ATA production. In particular, it is aimed to avoid or minimize false-positive results in ATA-diagnostic assays.

It was found that these goals can be achieved by the use of a radioimmunoassay which can be applied as a diagnostic method for determining the presence of antigen-specific IgG antibodies. This type of test, which is designated as “antigen binding test” (6), is based on the use of a solid carrier which is capable of binding the constant region of IgG antibodies, and comprises the following steps:

a) providing a solid carrier capable of binding the constant region of IgG antibodies,
b) isolating a sample from a subject to be tested for the presence of IgG antibodies against a therapeutic antibody,
c) incubating said carrier with said sample (e.g. a patient serum sample) under conditions suitable for immobilizing IgG antibodies on said solid carrier, typically followed by one or more washing steps, in order to remove unbound serum components,
d) contacting said immobilized antibodies with an antigenic fragment of said therapeutic antibody under conditions that allow for complex formation between at least part of said immobilized antibodies and said antigenic fragment, said fragment lacking a constant region and being conjugated to a detectable label; and wherein said contacting is performed in the presence of) an unlabeled antigenic fragment of a non-therapeutic antibody, and, after a washing step to remove unbound labelled fragment, and
e) detecting the amount of detectable label in the complex to indicate the presence of IgG antibodies against a therapeutic antibody in the sample.

When the total IgG response against TA are tested by using an immobilized anti-IgG reagent which binds all subclasses, steps c) and d) may be combined

In this assay format, antibodies of relevant IgG-types are captured and immobilized, including “true” ATA's as well as those displaying reactivity with antigenic fragments irrespective of the specificity of the fragments, e.g. F(ab′)2 fragments showing up as “false” ATA's. However, in the present assay the subsequent binding of labelled antigenic fragment to the “false” ATA is significantly reduced by the presence of unlabeled competitor fragment.

A method of the invention can thus be used for monitoring and quantitating the presence of various IgG-type ATA's. Generally speaking, most of the IgG antibodies against a therapeutic antibody are IgG4 and/or IgG1 type antibodies. It should be noted that, using this test format, in principle specific antibodies of all immunoglobulin classes can be tested, depending on the type of immobilized antibody-catching components on the solid phase.

Step a) of the method comprises the use of a solid carrier capable of binding the constant region of IgG antibodies. The solid carrier is for example agarose, cellulose, dextran, Sephadex, Sepharose, carboxymethyl cellulose, polystyrene, filter paper, ion-exchange resin, plastic, glass, nylon, silk, etc. The carrier may be in the shape of, for example, a sheet, a tube, a test plate, bead, disc, sphere, and the like.

Materials capable of binding the constant region of IgG antibodies (Fc) are known in the art. They include protein A, Protein G and antibodies against IgG, such as monoclonal antibodies, polyclonal antibodies and single chain recombinant antibodies. The IgG-binding material can be attached to the solid carrier by any suitable means, e.g. chemical or enzymatic coupling.

In one typical embodiment, the solid carrier is a carrier comprising Sepharose-coupled protein A or protein G; both carriers are commercially available as protein A-Sepharose or protein G-Sepharose. The sample taken will generally be a biological fluid comprising antibodies (some of which may be ATA's), such as a serum sample that can be prepared according to standard practise from a blood sample of the subject.

It has been observed that some serum samples contain IgG antibodies which bind F(ab′)2 antibodies (and therefore also the labelled antigen fragment) in a non-specific manner, resulting in false-positive test results.

Step b) involves the preparation of a sample isolated from a subject to be tested, typically a patient receiving TA therapy or a test subject participating in a clinical TA trial. The sample taken will generally be a biological fluid comprising antibodies (some of which may be ATA's), such as a serum sample that can be prepared according to standard practise from a blood sample of the subject.

Step c) involves incubating the IgG-binding carrier prepared in step a) with at least part of the sample prepared in step b) under conditions that allow for immobilizing IgG antibodies on said solid carrier. The skilled person will know the conditions that are suitable and also which conditions to are to be avoided, e.g. high salt concentrations and/or extreme pH values could interfere with the interaction between antibodies and IgG-binding solid carrier. The incubation is preferably performed in a buffer, for example a buffer in the pH range of 7.2-7.6 and comprising one or more salts, such as phosphate-buffered saline (PBS), Tween0.2% (v/v); EDTA 0.01 M, NaN3 (0.05% (w/v); BSA 0.3%; (PBS-AT). Incubation can be performed at different temperatures and during different time periods. Suitable temperatures range for example from about 4° C. to about room temperature. The incubation is generally performed during several hours, e.g. 2-48 hours to allow for immobilizing IgG antibodies on the solid carrier. The reaction mixture may be incubated under agitation, for instance using a rotator to ensure efficient mixing of the solid carrier and the sample.

As will be understood by the skilled person, sufficient binding capacity of the solid phase is important for obtaining relevant test results. The relative amounts of carrier and test sample to be incubated can vary, depending for instance on the type of sample and/or the carrier used. In one embodiment, 50 μl of 1/50 diluted serum sample is contacted with 1 mg agarose-immobilized protein A in a total volume of 750 μl. In another embodiment a 50 μl 1/50 diluted serum sample sample is contacted with 0.5 ml suspension of 1 mg/ml carrier, e.g. Sepharose-coupled antibodies to IgG4 .

Following the immobilization of the IgG antibodies on the solid carrier, some of which may be ATA's, the immobilized antibodies are incubated in step d) of the assay according to the invention with an antigenic fragment of the therapeutic antibody of interest, said fragment lacking a constant region and being conjugated to a detectable label. In other words, use is made of a labelled antigenic fragment of the therapeutic antibody that may have caused ATA production, which fragment is readily detectable yet uncapable of binding to the solid carrier prepared in step a). The labelled antigenic fragment can be recognized by and bound to an immobilized ATA. Again, conditions are used that allow for complex formation between at least part of said immobilized antibodies and said labelled antigenic fragment.

Step e) involves detecting the amount of detectable label in the complex formed between immobilized antibodies and antigenic fragment to indicate the presence of IgG antibodies against a therapeutic antibody in the sample. Thus, detecting the amount of labelled antigenic fragment that is specifically associated with the solid carrier in step e) provides an indication of the amount of ATA's originally present in the sample.

According to one embodiment of the invention, the incubation in the presence of irrelevant F(ab′)2 is characterized in that it reduces a possibly false positive outcome of the assay. The unlabeled antigenic fragment lacks a constant region and thus cannot bind directly to the IgG-binding solid carrier. The unlabeled antigenic fragment of a non-therapeutic antibody can minimize, or even avoid, the binding of labelled fragment to those immobilized'IgG antibodies that can non-specifically react with antigenic fragments irrespective of the specificity of the antigenic fragment, and therefore do not qualify as ATA. In a preferred embodiment, said labelled and/or unlabelled antigenic fragments are irrelevant F(ab′)2 fragments.

The labeled antigenic fragment lacking a constant region is suitably generated by protease treatment of the therapeutic antibody of interest according to established procedures. For example, elastase, trypsin, ficin, pepsin or papain can be used to, remove the constant region and release the antigen binding fragment.

Pepsin is commonly used in the preparation of F(ab′)2 fragments from antibodies. To produce a F(ab′)2 fragment, IgG is digested with pepsin, which cleaves the heavy chains near the hinge region. One or more of the disulfide bonds that join the heavy chains in the hinge region are preserved, so the two Fab regions of the antibody remain joined together, yielding a divalent molecule (containing two antibody binding sites), hence the designation F(ab′)2. The light chains remain intact and attached to the heavy chain. The Fc fragment is digested into small peptides. Protocols for antibody digestion and purification of antibody fragments can be found in (7).

Commercial kits are available for digesting antibodies into F(ab′)2 fragments that retain antigen binding activity. The protease ficin was found to be particularly suitable for the production of F(ab′)2 fragments from murine IgG1 (8).

To allow for detection of the immobilized ATA's, the antigenic fragment of the TA is provided with at least one detectable label. Any type of suitable label may be used. In one embodiment, the antigenic fragment is provided with a label selected from the group consisting of radioactive labels (e.g. ¹²⁵I), fluorescent chemicals (e.g. europium cryptate), colorimetric labels, and enzyme labels (e.g. horse radish peroxidase).

The label can be conjugated to the antigenic fragment using standard procedures. For example, direct radioiodination of Fab or F(ab′)2 fragments can be performed by the chloramine T method (9).

As is clear form the above, in a method of the invention the unlabeled antigenic fragment of a non-therapeutic antibody acts as “competitor” of the labelled fragment of the TA with respect to binding to immobilized antibodies. The competitor can be a F(ab′)2 fragment of mono- or polyclonal origin, for example obtained by protease treatment of a composition comprising IgG antibodies. A suitable IgG-comprising composition for preparing competitor fragments is InfraVenous Immunoglobulin (IVIG, Sanquin, The Netherlands). IVIG is a commercially available plasma-derived solution of globulins containing antibodies normally present in adult human blood. IVIG is used in many different autoimmune disorders, and most IVIG is produced from pooled human plasma derived from multiple blood donors. IVIG typically contains more than 95 percent unmodified IgG with intact immune signaling functions along with trace amounts of IgA and IgM, cytokines, soluble complement, and HLA molecules.

Again, useful proteases comprise elastase, trypsin, pepsin and papain. However, in a preferred embodiment, the protease used to obtain the unlabeled antigenic “competitor” fragment is the same protease as the one used to generate the labelled antigenic fragment of the therapeutic antibody. Because of the similar protease treatment, the chances are increased that non-specific recognition of a labelled fragment by an antibody (causing a false-positive signal) is efficiently blocked by a similar, non-labeled fragment.

The skilled person will understand that the present invention can be applied to detect antibodies against any type of therapeutic antibody. Most of them are monoclonal antibodies (mAb), chimeric or ‘humanized’ antibodies, or fragments thereof. The therapeutic antibody is for example an anti-cancer antibody or an anti-inflammatory antibody. For example, the therapeutic antibody is used for the treatment of (auto)immune disease, including rheumatoid arthritis (RA), Crohn's disease, Kawasaki syndrome, allergic disorders etc.

In a specific aspect, there is provided a diagnostic method for determining the presence of IgG antibodies against a therapeutic anti-TNFα antibody in a subject, comprising the steps of:

a) providing a solid carrier capable of binding the constant region of IgG antibodies,
b) isolating a sample from a subject to be tested for the presence of IgG antibodies against an anti-TNFα antibody therapeutic antibody, for instance a patient suffering from RA and receiving infliximab or a similar therapeutic antibody;
c) incubating said carrier with said test sample under conditions suitable for immobilizing IgG antibodies on said solid carrier,
d) incubating said immobilized antibodies with an antigenic fragment of the anti-TNFα antibody lacking a constant region and being conjugated to a detectable label, e.g. ¹²⁵I-labeled pepsin-treated Infliximab; and wherein said incubating is performed in the presence of an unlabeled antigenic fragment (irrelevant F(ab′)2 fragment) of a non-therapeutic antibody lacking a constant region, e.g. pepsin-treated IVIG; and
c) detecting the amount of ¹²⁵I in the complex to indicate the presence of IgG antibodies against the anti-TNFα antibody therapeutic antibody in the sample.

The invention also provides diagnostic kits for use in a method according to the invention, said kits being characterized in that they comprise at least a labeled antigenic fragment of a therapeutic antibody and an unlabeled F(ab′)2 fragment of a non-therapeutic antibody.

The labelled antigenic fragment of a therapeutic antibody and said unlabeled antigenic fragment of a non-therapeutic antibody can be present in a single container (e.g. lyophilized with buffer salts). In one embodiment, a kit comprises a buffer containing Tween0.2% (v/v); EDTA 0.01 M, NaN3 (0.05% (w/v); BSA 0.3% and IVIG-F(ab′)2 (10 μg/ml).

The kit may furthermore comprise a solid carrier capable of binding the constant region of IgG antibodies, for example beads, paper or plastic surface provided with at least one IgG-binding component, preferably selected from the group consisting of protein A, Protein G, monoclonal antibodies, polyclonal antibodies and single chain recombinant antibodies against IgG. In one embodiment, the kit comprises a suspension of Sepharose-coupled protein A, protein G or Sepharose-coupled antibodies against IgG4 .

In one embodiment, the unlabeled antigenic fragment of a non-therapeutic antibody in the kit is a F(ab′)2 fragment of mono- or polyclonal origin, optionally obtained from protease treatment of purified IgG (e.g. IVIG)

As indicated above, the antigenic fragments are easily obtainable by protease treatment of (non)-therapeutic antibody. Suitable proteases include elastase, trypsin, pepsin and papain, and more preferably pepsin. It may be advantageous that the labelled antigenic fragment of a therapeutic antibody and an unlabeled antigenic fragment of a non-therapeutic antibody contained in the diagnostic are prepared using the same protease treatment.

The fragment of the therapeutic antibody in the kit can be a fragment of any therapeutic antibody of interest, i.e. a therapeutic antibody suspected or known to induce the formation of antibodies in a patient. Provided is a diagnostic kit for determining the presence of IgG(4) antibodies against a therapeutic antibody in a subject, wherein said therapeutic antibody is an anti-cancer antibody or an anti-inflammatory antibody.

Other components of the kit may include one or more component(s) selected from the group consisting of a positive reference samples, a negative reference, dilution buffer, instructions for use. The positive and negative reference samples are suitably used to verify that the assay has been properly performed. Also, they may serve to convert the detected signal to (non)-arbitrary units, e.g. AU/ml or μg ATA/ml serum.

Also provided herein is the use of a method and/or a kit according to the invention to optimize therapeutic antibody treatment of a subject. Optimization may be performed in a clinical or experimental setting.

### LEGEND TO THE FIGURES

FIG. 1: Results obtained when the assay according to the invention was used to detect the presence of HACA (antibodies against infliximab) in blood donor sample known to be HACA-negative. Open bars represent the data obtained using a reference incubation buffer not containing irrelevant F(ab′)2 as competitor. Filled bars represent the data when the assay was performed in the presence of IVIG F(ab′)2. Y-axis indicates amount of binding of ¹²⁵I-radiolabelled F(ab′)2 fragment and is indicative for the presence of HACA. For details see Example 1.

FIG. 2: Serum samples from patients A through M who were treated with adalimumab were tested for HAHA (antibodies against adalimumab). Patients A through J showed no clinical abnormalities, in contrast to patients K, L and M. Open bars represent the data obtained using a reference incubation buffer not containing irrelevant F(ab′)2 as competitor. Filled bars represent the data when the assay was performed in the presence of IVIG F(ab′)2. Y-axis indicates amount of binding of ¹²⁵I-radiolabelled F(ab′)2 fragment and is indicative for the presence of HAHA. For details see Example 1.

The invention is exemplified by the Examples below.

### EXAMPLE 1

### Protocol

50 μl serum dilution (1/50 in PBS-AT) is incubated for 16 hours with 500 μl ProteinA Sepharose suspension (2 mg/ml in PBS-AT) in a 4 ml tube on a rotator.
Non-bound serum components are washed away by spinning down the Seharose and removal of the supernatant (5 times)
50 μl of ¹²⁵I-radiolabelled F(ab′)2 fragment (corresponding to approximately 1 ng of radiolabelled protein in PBS-AT), is added, followed by addition of 500 μl of incubation buffer according to the invention (PBS-AT, 10 μg IVIG F(ab′)2/ml). The tube is incubated over night on a rotator
Non-bound radiolabel is washed away by spinning down the Sepharose and removal of the supernatant (5 times)
Binding of radiolabel is determined by gamma counting, and test results are quantified by application of a serially diluted standard

### Results

The method of the invention was evaluated in an assay to detect HACA (antibodies against infliximab) or HAHA (antibodies against adalimumab).

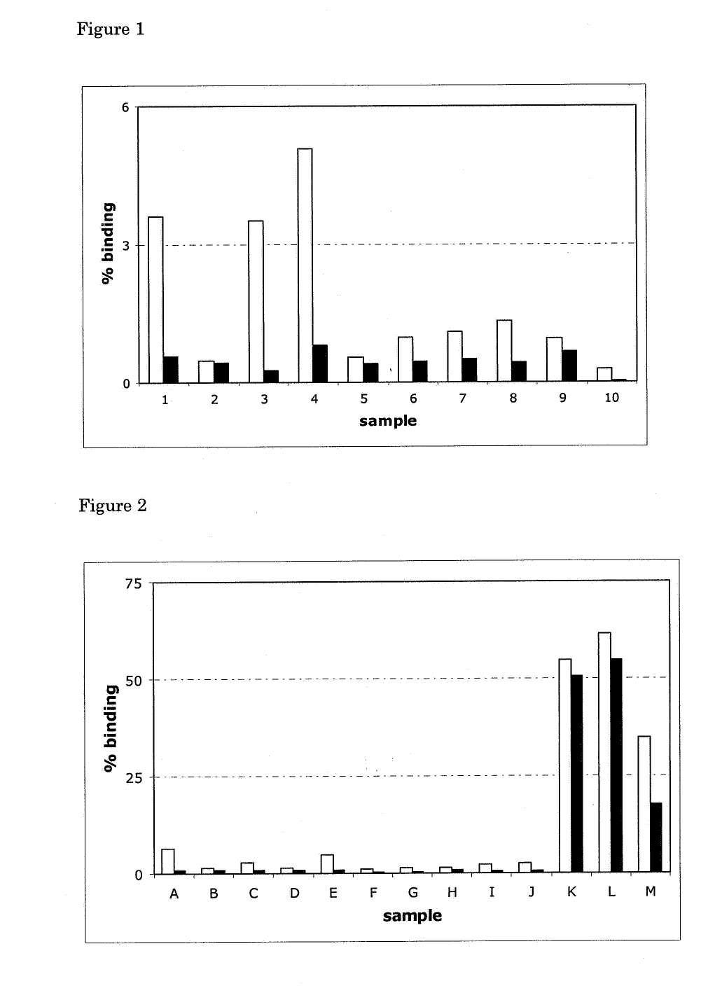

FIG. 1 shows the results obtained when the assay according to the invention was used to detect the presence of HACA. Open bars represent the data obtained using a reference incubation buffer not containing irrelevant F(ab′)2 as competitor. Three out of nine sera would erroneously have been designated as HACA-positive (>1% binding). Filled bars represent the reduction in false positive results in donors when the assay was performed in the presence of IVIG F(ab′)2 .

A similar phenomenon was observed when serum samples from patients who were treated with adalimumab were tested for HAHA (see FIG. 2). Samples show clearly positive results in the absence of competitor fragment, wherein these patient show no clinical abnormalities (A, C, D). Typical patients (K, L, M) give, when tested according to the invention, clearly positive test results.

### Conclusion:

Testing for ATA's. such as HACA and HACA, in the presence of an unlabeled antigenic fragment of a non-therapeutic antibody lacking a constant region greatly enhances the reliability of immunogenicity testing.

### EXAMPLE 2

### Composition of a Test Kit

A test kit comprises labelled F(ab′)2 fragment from the therapeutic monoclonal antibody concerned, as well as F(ab′)2 fragment obtained from a preparation of irrelevant antibodies from the same antibody class as the therapeutic antibody concerned, generally IgG. Apart from that, the kit may contain immobilized reagents, which are able to catch serum antibodies of interest, and washing buffer.

### REFERENCES

1. Holliger et al. (2005) Nature Biotech., 23:1126-1136
2. Theillaud (2005) Expert Opin. Biol. Ther., 5 (Suppl. 1):S15-S27
3. Baert et al. (2003) New Engl J Med 348:601
4. Wolbink et al. (2006) Arthritis & Rheumatism, Vol. 54, No. 3, pp. 711-715
5. Current Pharmaceutical Biotechnology 3, 349-360, 2002
6. Aalberse et al. (1983) J Immunol 1983; 130:722-6
7. Antibodies: A Laboratory Manual, E. Harlow and D. Lane, ed., Cold Spring Harbor Laboratory, Cold Spring Harbor, N.Y., 1988 (A2926)
8. Mariani, M. et al. (1991). Mol. Immunol. 28, 69-77
9. Arano et al. (1996) Bioconjugate Chem., 7: 628-637

1. A diagnostic method for determining the presence of IgG antibodies against a therapeutic antibody in a subject, comprising the steps of:
a) providing a solid carrier capable of binding the constant region (Fc) of IgG antibodies,
b) isolating a sample from a subject to be tested for the presence of IgG antibodies against a therapeutic antibody,
c) incubating said carrier with said sample under conditions suitable for immobilizing IgG antibodies on said solid carrier;
d) incubating said immobilized antibodies with an antigenic fragment of said therapeutic antibody under conditions that allow for complex formation between at least part of said immobilized antibodies and said antigenic fragment, said fragment lacking a constant region (Fc) and being conjugated to a detectable label;
and wherein said incubation is performed in the presence of an unlabeled antigenic fragment of a non-therapeutic antibody lacking a constant region (Fc), and
e) detecting the amount of detectable label in the complex to indicate the presence of IgG antibodies against a therapeutic antibody in the sample.

2. Method according to claim 1, wherein said IgG antibodies against a therapeutic antibody are IgG4 and/or IgG1 antibodies.

3. Method according to claim 1, wherein said solid carrier is a bead, such as a Sepharose bead or a magnetic bead, a paper or plastic surface.

4. Method according to claim 1, wherein said solid carrier carries at least one IgG-binding component selected from the group consisting of protein A, Protein G, monoclonal antibodies, polyclonal antibodies and single chain recombinant antibodies against IgG.

5. Method according to claim 1 wherein said antigenic fragments are F(ab′)2 fragments.

6. Method according to claim 1 wherein said antigenic fragment is generated by protease treatment of therapeutic antibody, preferably selected from elastase, trypsin, pepsin and papain, more preferably pepsin.

7. Method according to claim 1, wherein said detectable label is a radioactive label, a fluorophore or an enzyme.

8. Method according to claim 1 wherein said unlabeled antigenic fragment of a non-therapeutic antibody is a F(ab)2 fragment of mono- or polyclonal origin, optionally obtained from protease treatment of a composition comprising IgG (IVIg or IgG-containing serum).

9. Method according to claim 8, wherein said protease is selected from elastase, trypsin, pepsin and papain, preferably wherein the protease used to obtain unlabeled antigenic fragment is the same protease used to generate the labelled antigenic fragment of the therapeutic antibody.

10. Method according to claim 1, wherein said therapeutic antibody is an anti-cancer antibody or an anti-inflammatory antibody, preferably wherein said therapeutic antibody is an anti-TNFα antibody.

11. Method according to claim 1, wherein said subject is a human subject being or having been treated with a therapeutic antibody, preferably a human subject suffering from cancer or an (auto)immune disease, including rheumatoid arthritis (RA), Crohn's disease, Kawasaki syndrome, or from allergic disorders.

12. A diagnostic kit for use in a method according to claim 1, said kit comprising a labeled antigenic fragment of a therapeutic antibody and an unlabeled antigenic fragment of a non-therapeutic antibody, said antigenic fragments lacking a constant region (Fc).

13. Kit according to claim 12, wherein said labelled antigenic fragment of a therapeutic antibody and said unlabeled antigenic fragment of a non-therapeutic antibody are present in a single container.

14. Kit according to claim 12, furthermore comprising a solid carrier capable of binding the constant region (Fc) of IgG antibodies.

15. Kit according to claim 14, wherein said solid carrier is a bead, such as a Sepharose bead or a magnetic bead, a paper or plastic surface.

16. Kit according to claim 12, wherein said antigenic fragment is generated by protease treatment of therapeutic antibody, preferably selected from elastase, trypsin, pepsin and papain, more preferably pepsin.

17. Kit according to claim 12, wherein said detectable label is a radioactive label, a fluorophore or an enzyme.

18. Kit according to claim 12, wherein said unlabeled antigenic fragment of a non-therapeutic antibody is a F(ab)2 fragment of mono- or polyclonal origin, optionally obtained from protease treatment of a composition comprising IgG (IVIg or IgG-containing serum).

19. Kit according to claim 18, wherein said protease is selected from elastase, trypsin, pepsin and papain, preferably wherein the protease used to obtain unlabeled antigenic fragment is the same protease used to generate the labelled antigenic fragment of the therapeutic antibody.

20. Kit according to any claim 12, wherein said therapeutic antibody is an anti-cancer antibody or an anti-inflammatory antibody, preferably wherein said therapeutic antibody is an anti-TNFα antibody.

21. Use of a method according to claim 1 to optimize therapeutic antibody treatment of a subject.

22. Use of a kit according to claim 12 to optimize therapeutic antibody treatment of a subject.
